SEQUENCE LISTING (85 sequences)

SEQ ID NO: 1 - protein, 122 aa, Synthetic construct
QVQLQESGGGLVQAGGSLRLSCTASERTFSDYALGWFRQGPGKEREFVAAINWVGGSTYYADSVKGRFTISRDNAKNTLLLQMNSLKPEDTAVYFCVASWGAMPVLTHVINYWGKGTQVTVS

SEQ ID NO: 2 - protein, 8 aa, Synthetic construct
ERTFSDYA

SEQ ID NO: 3 - protein, 8 aa, Synthetic construct
INWVGGST

SEQ ID NO: 4 - protein, 16 aa, Synthetic construct
VASWGAMPVLTHVINY

SEQ ID NO: 5 - protein, 123 aa, Synthetic construct
QVQLQESGGGLVQAGGSLRLSCTASERTFSDYALGWFRQGPGKEREFVAAINWVGGSTYYADSVKGRFTISRDNAKNTLLLQMNSLKPEDTAVYFCVASWGAMPVLTHVINYWGKGTQVTVSS

SEQ ID NO: 6 - protein, 8 aa, Synthetic construct
ERTFSDYA

SEQ ID NO: 7 - protein, 8 aa, Synthetic construct
INWVGGST

SEQ ID NO: 8 - protein, 16 aa, Synthetic construct
VASWGAMPVLTHVINY

SEQ ID NO: 9 - protein, 123 aa, Synthetic construct
QVQLVESGGGLVQPGGSLRLSCSASERTFSDYAMHWVRQAPGKGLEYVSAINWVGGSTYYADSVKGRFTISRDNSKNTLYLQMNSLRAEDTAVYYCVASWGAMPVLTHVINYWGQGTLVTVSS

SEQ ID NO: 10 - protein, 8 aa, Synthetic construct
ERTFSDYA

SEQ ID NO: 11 - protein, 8 aa, Synthetic construct
INWVGGST

SEQ ID NO: 12 - protein, 16 aa, Synthetic construct
VASWGAMPVLTHVINY

SEQ ID NO: 13 - protein, 123 aa, Synthetic construct
QVQLVESGGGLVQPGGSLRLSCSASERTFSDYAMHWFRQAPGKEREYVSAINWVGGSTYYADSVKGRFTISRDNSKNTLYLQMNSLRAEDTAVYYCVASWGAMPVLTHVINYWGQGTLVTVSS

SEQ ID NO: 14 - protein, 8 aa, Synthetic construct
ERTFSDYA

SEQ ID NO: 15 - protein, 8 aa, Synthetic construct
INWVGGST

SEQ ID NO: 16 - protein, 16 aa, Synthetic construct
VASWGAMPVLTHVINY

SEQ ID NO: 17 - protein, 123 aa, Synthetic construct
QVQLVESGGGLVQPGGSLRLSCSASERTFSDYAMGWFRQAPGKEREYVSAINWVGGSTYYADSVKGRFTISRDNSKNTLYLQMNSLRAEDTAVYYCVASWGAMPVLTHVINYWGQGTLVTVSS

SEQ ID NO: 18 - protein, 8 aa, Synthetic construct
ERTFSDYA

SEQ ID NO: 19 - protein, 8 aa, Synthetic construct
INWVGGST

SEQ ID NO: 20 - protein, 16 aa, Synthetic construct
VASWGAMPVLTHVINY

SEQ ID NO: 21 - protein, 123 aa, Synthetic construct
QVQLVESGGGLVQPGGSLRLSCSASERTFSDYAMHWFRQAPGKEREFVSAINWVGGSTYYADSVKGRFTISRDNSKNTLYLQMNSLRAEDTAVYYCVASWGAMPVLTHVINYWGQGTLVTVSS

SEQ ID NO: 22 - protein, 8 aa, Synthetic construct
ERTFSDYA

SEQ ID NO: 23 - protein, 8 aa, Synthetic construct
INWVGGST

SEQ ID NO: 24 - protein, 16 aa, Synthetic construct
VASWGAMPVLTHVINY

SEQ ID NO: 25 - protein, 123 aa, Synthetic construct
QVQLVESGGGLVQPGGSLRLSCSASERTFSDYAMGWFRQAPGKEREFVSAINWVGGSTYYADSVKGRFTISRDNSKNTLYLQMNSLRAEDTAVYYCVASWGAMPVLTHVINYWGQGTLVTVSS

SEQ ID NO: 26 - protein, 8 aa, Synthetic construct
ERTFSDYA

SEQ ID NO: 27 - protein, 8 aa, Synthetic construct
INWVGGST

SEQ ID NO: 28 - protein, 16 aa, Synthetic construct
VASWGAMPVLTHVINY

SEQ ID NO: 29 - protein, 123 aa, Synthetic construct
QVQLVESGGGLVQPGGSLRLSCSASERTFSDYAMGWFRQAPGKEREFVAAINWVGGSTYYADSVKGRFTISRDNSKNTLYLQMNSLRAEDTAVYYCVASWGAMPVLTHVINYWGQGTLVTVSS

SEQ ID NO: 30 - protein, 8 aa, Synthetic construct
ERTFSDYA

SEQ ID NO: 31 - protein, 8 aa, Synthetic construct
INWVGGST

SEQ ID NO: 32 - protein, 16 aa, Synthetic construct
VASWGAMPVLTHVINY

SEQ ID NO: 33 - protein, 123 aa, Synthetic construct
QVQLVESGGGLVQPGGSLRLSCSASERTFSDYAMGWFRQAPGKEREFVSAINWVGGSTYYADSVKGRFTISRDNSKNTLYLQMNSLRAEDTAVYYCVASWGAMPVLTHVINYWGKGTLVTVSS

SEQ ID NO: 34 - protein, 8 aa, Synthetic construct
ERTFSDYA

SEQ ID NO: 35 - protein, 8 aa, Synthetic construct
INWVGGST

SEQ ID NO: 36 - protein, 16 aa, Synthetic construct
VASWGAMPVLTHVINY

SEQ ID NO: 37 - protein, 123 aa, Synthetic construct
QVQLVESGGGLVQPGGSLRLSCSASERTFSDYAMGWFRQAPGKEREFVAAINWVGGSTYYADSVKGRFTISRDNSKNTLYLQMNSLRAEDTAVYYCVASWGAMPVLTHVINYWGKGTLVTVSS

SEQ ID NO: 38 - protein, 8 aa, Synthetic construct
ERTFSDYA

SEQ ID NO: 39 - protein, 8 aa, Synthetic construct
INWVGGST

SEQ ID NO: 40 - protein, 16 aa, Synthetic construct
VASWGAMPVLTHVINY

SEQ ID NO: 41 - protein, 123 aa, Synthetic construct
QVQLVESGGGLVQPGGSLRLSCSASERTFSDYALGWFRQAPGKEREFVAAINWVGGSTYYADSVKGRFTISRDNSKNTLYLQMNSLRAEDTAVYYCVASWGAMPVLTHVINYWGKGTLVTVSS

SEQ ID NO: 42 - protein, 8 aa, Synthetic construct
ERTFSDYA

SEQ ID NO: 43 - protein, 8 aa, Synthetic construct
INWVGGST

SEQ ID NO: 44 - protein, 16 aa, Synthetic construct
VASWGAMPVLTHVINY

SEQ ID NO: 45 - protein, 123 aa, Synthetic construct
QVQLQESGGGLVQPGGSLRLSCSASERTFSDYALGWFRQAPGKEREFVAAINWVGGSTYYADSVKGRFTISRDNSKNTLYLQMNSLRAEDTAVYYCVASWGAMPVLTHVINYWGKGTLVTVSS

SEQ ID NO: 46 - protein, 8 aa, Synthetic construct
ERTFSDYA

SEQ ID NO: 47 - protein, 8 aa, Synthetic construct
INWVGGST

SEQ ID NO: 48 - protein, 16 aa, Synthetic construct
VASWGAMPVLTHVINY

SEQ ID NO: 49 - protein, 123 aa, Synthetic construct
QVQLVESGGGLVQAGGSLRLSCSASERTFSDYALGWFRQAPGKEREFVAAINWVGGSTYYADSVKGRFTISRDNSKNTLYLQMNSLRAEDTAVYYCVASWGAMPVLTHVINYWGKGTLVTVSS

SEQ ID NO: 50 - protein, 8 aa, Synthetic construct
ERTFSDYA

SEQ ID NO: 51 - protein, 8 aa, Synthetic construct
INWVGGST

SEQ ID NO: 52 - protein, 16 aa, Synthetic construct
VASWGAMPVLTHVINY

SEQ ID NO: 53 - protein, 123 aa, Synthetic construct
QVQLVESGGGLVQPGGSLRLSCTASERTFSDYALGWFRQAPGKEREFVAAINWVGGSTYYADSVKGRFTISRDNSKNTLYLQMNSLRAEDTAVYYCVASWGAMPVLTHVINYWGKGTLVTVSS

SEQ ID NO: 54 - protein, 8 aa, Synthetic construct
ERTFSDYA

SEQ ID NO: 55 - protein, 8 aa, Synthetic construct
INWVGGST

SEQ ID NO: 56 - protein, 16 aa, Synthetic construct
VASWGAMPVLTHVINY

SEQ ID NO: 57 - protein, 123 aa, Synthetic construct
QVQLVESGGGLVQPGGSLRLSCSASERTFSDYALGWFRQGPGKEREFVAAINWVGGSTYYADSVKGRFTISRDNSKNTLYLQMNSLRAEDTAVYYCVASWGAMPVLTHVINYWGKGTLVTVSS

SEQ ID NO: 58 - protein, 8 aa, Synthetic construct
ERTFSDYA

SEQ ID NO: 59 - protein, 8 aa, Synthetic construct
INWVGGST

SEQ ID NO: 60 - protein, 16 aa, Synthetic construct
VASWGAMPVLTHVINY

SEQ ID NO: 61 - protein, 123 aa, Synthetic construct
QVQLVESGGGLVQPGGSLRLSCSASERTFSDYALGWFRQAPGKEREFVAAINWVGGSTYYADSVKGRFTISRDNAKNTLYLQMNSLRAEDTAVYYCVASWGAMPVLTHVINYWGKGTLVTVSS

SEQ ID NO: 62 - protein, 8 aa, Synthetic construct
ERTFSDYA

SEQ ID NO: 63 - protein, 8 aa, Synthetic construct
INWVGGST

SEQ ID NO: 64 - protein, 16 aa, Synthetic construct
VASWGAMPVLTHVINY

SEQ ID NO: 65 - protein, 123 aa, Synthetic construct
QVQLVESGGGLVQPGGSLRLSCSASERTFSDYALGWFRQAPGKEREFVAAINWVGGSTYYADSVKGRFTISRDNSKNTLLLQMNSLRAEDTAVYYCVASWGAMPVLTHVINYWGKGTLVTVSS

SEQ ID NO: 66 - protein, 8 aa, Synthetic construct
ERTFSDYA

SEQ ID NO: 67 - protein, 8 aa, Synthetic construct
INWVGGST

SEQ ID NO: 68 - protein, 16 aa, Synthetic construct
VASWGAMPVLTHVINY

SEQ ID NO: 69 - protein, 123 aa, Synthetic construct
QVQLVESGGGLVQPGGSLRLSCSASERTFSDYALGWFRQAPGKEREFVAAINWVGGSTYYADSVKGRFTISRDNSKNTLYLQMNSLKPEDTAVYYCVASWGAMPVLTHVINYWGKGTLVTVSS

SEQ ID NO: 70 - protein, 8 aa, Synthetic construct
ERTFSDYA

SEQ ID NO: 71 - protein, 8 aa, Synthetic construct
INWVGGST

SEQ ID NO: 72 - protein, 16 aa, Synthetic construct
VASWGAMPVLTHVINY

SEQ ID NO: 73 - protein, 123 aa, Synthetic construct
QVQLVESGGGLVQPGGSLRLSCSASERTFSDYALGWFRQAPGKEREFVAAINWVGGSTYYADSVKGRFTISRDNSKNTLYLQMNSLRAEDTAVYFCVASWGAMPVLTHVINYWGKGTLVTVSS

SEQ ID NO: 74 - protein, 8 aa, Synthetic construct
ERTFSDYA

SEQ ID NO: 75 - protein, 8 aa, Synthetic construct
INWVGGST

SEQ ID NO: 76 - protein, 16 aa, Synthetic construct
VASWGAMPVLTHVINY

SEQ ID NO: 77 - protein, 123 aa, Synthetic construct
QVQLVESGGGLVQPGGSLRLSCSASERTFSDYALGWFRQAPGKEREFVAAINWVGGSTYYADSVKGRFTISRDNSKNTLYLQMNSLRAEDTAVYYCVASWGAMPVLTHVINYWGKGTQVTVSS

SEQ ID NO: 78 - protein, 8 aa, Synthetic construct
ERTFSDYA

SEQ ID NO: 79 - protein, 8 aa, Synthetic construct
INWVGGST

SEQ ID NO: 80 - protein, 16 aa, Synthetic construct
VASWGAMPVLTHVINY

SEQ ID NO: 81 - protein, 20 aa, Homo sapiens
MEAPAQLLFLLLLWLPDTTG

SEQ ID NO: 82 - protein, 232 aa, Homo sapiens
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 107 aa, Homo sapiens [sequence omitted]

SEQ ID NO: 85 - protein, 7 aa, Synthetic construct
HHHHHHC

(19) United States
(12) Patent Application Publication
(10) Pub. No.: US 2026/0284212 A1
(43) Pub. Date: Sep. 24, 2026

(54) HUMAN CD6 BINDING MOLECULES

(71) Applicant: THE CLEVELAND CLINIC FOUNDATION, Cleveland, OH (US)

(72) Inventor: Feng LIN, Cleveland, OH (US)

(21) Appl. No.: 19/167,924

(22) PCT Filed: Mar. 22, 2024
(86) PCT No.: PCT/US2024/021161

§ 371 Date: Sep. 23, 2025

Related U.S. Application Data
(60) Provisional application No. 63/454,127, filed on Mar. 23, 2023.

(51) Int. Cl. A61K 47/68; A61P 35/00; C07K 16/28; G01N 33/575

(52) U.S. Cl. CPC A61K 47/68031; A61K 47/6849; A61P 35/00; C07K 16/2896; G01N 33/5759; C07K 2317/24; C07K 2317/524; C07K 2317/526; C07K 2317/565; C07K 2317/567; C07K 2317/569; G01N 2333/70596

(57) ABSTRACT

Provided herein are human Cluster of Differentiation 6 (CD6) binding molecules and nucleic acid sequences encoding such molecules. In particular embodiments, provided herein are human CD6 binding molecules (e.g., nanobodies) having a first, and optionally a second, single monomeric variable antibody domain (SMVAD) that comprises certain CDRs, and methods for using such molecules to treat T-cell related diseases (e.g., cancer, such as T-cell lymphoma). In certain embodiments, the SMVAD comprises camelid, human, or humanized framework regions.

The present application claims priority to U.S. Provisional application Ser. No. 63/454,127 filed Mar. 23, 2023, which is herein incorporated by reference in its entirety.

This invention was made with government support under EY025373 awarded by the National Institutes of Health. The government has certain rights in the invention.

### SEQUENCE LISTING

The text of the computer readable sequence listing filed herewith titled “CCF_41787_601_SequenceListing.xml”, created on Mar. 22, 2024, having a file size of 77,551 bytes, is hereby incorporated by reference in its entirety.

### FIELD OF THE INVENTION

Provided herein are human Cluster of Differentiation 6 (CD6) binding molecules and nucleic acid sequences encoding such molecules. In particular embodiments, provided herein are human CD6 binding molecules (e.g., nanobodies) having a first, and optionally a second, single monomeric variable antibody domain (SMVAD) that comprises certain CDRs, and methods for using such molecules to treat T-cell related diseases (e.g., cancer, such as T-cell lymphoma). In certain embodiments, the SMVAD comprises camelid, human, or humanized framework regions.

### BACKGROUND OF THE INVENTION

T-cell lymphoma is a rare form of cancerous lymphoma affecting T-cells. Lymphoma arises mainly from the uncontrolled proliferation of T-cells and can become cancerous. T-cell lymphoma is categorized under Non-Hodgkin lymphoma (NHL) and represents less than 15% of all Non-Hodgkin's diseases in the category. T-cell lymphomas are often categorized based on their growth patterns as either; aggressive (fast-growing) or indolent (slow-growing). Although the cause of T-cell lymphoma is not definitive, it has been associated with various risk factors and viruses such as Epstein-Barr virus (EBV) and Human T-cell leukemia virus-1 (HTLV1).

The prognosis and treatment of T-cell lymphoma can vary drastically based on the specific type of lymphoma and its growth patterns. Due to their rarity and high variability between the different subtypes, the prognosis of T-cell lymphoma is significantly worse than other Non-Hodgkin lymphoma. The treatment of T-cell lymphoma is often similar to other Non-Hodgkin lymphomas with early-stage treatments consisting of chemotherapy and/or radiology. The effectiveness of these treatments is often varied between subtypes with most receiving a poor outcome with high relapse rates.

### SUMMARY OF THE INVENTION

Provided herein are human Cluster of Differentiation 6 (CD6) binding molecules and nucleic acid sequences encoding such molecules. In particular embodiments, provided herein are human CD6 binding molecules (e.g., nanobodies) having a first, and optionally a second, single monomeric variable antibody domain (SMVAD) that comprises certain CDRs, and methods for using such molecules to treat T-cell related diseases (e.g., cancer, such as T-cell lymphoma). In certain embodiments, the SMVAD comprises camelid, human, or humanized framework regions. In some embodiments, the human CD6 binding molecules are human CD6 binding molecules.

In some embodiments, provided herein are compositions comprising a human Cluster of Differentiation 6 (CD6) binding molecule, or one or more nucleic acid molecules encoding said human CD6 binding molecule, wherein said human CD6 binding molecule comprises a first single monomeric variable antibody domain (SMVAD) that comprises: A) a CDR1 amino acid sequence comprising SEQ ID NO:2, 6, 10, 14, 18, 22, 26, 30, 34, 38, 42, 46, 50, 54, 58, 62, 66, 70, 74, or 78; or SEQ ID NO:2, 6, 10, 14, 18, 22, 26, 30, 34, 38, 42, 46, 50, 54, 58, 62, 66, 70, 74, or 78; with one with one or two conservative amino acid changes, B) a CDR2 amino acid sequence comprising SEQ ID NO:3, 7, 11, 15, 19, 23, 27, 31, 35, 39, 43, 47, 51, 55, 59, 63, 67, 71, 75, or 79; or SEQ ID NO:3, 7, 11, 15, 19, 23, 27, 31, 35, 39, 43, 47, 51, 55, 59, 63, 67, 71, 75, or 79 with one or two conservative amino acid changes, and C) a CDR3 amino acid sequence comprising SEQ ID NO:4, 8, 12, 16, 20, 24, 28, 32, 36, 40, 44, 48, 52, 56, 60, 64, 68, 72, 76, or 80; or SEQ ID NO:4, 8, 12, 16, 20, 24, 28, 32, 36, 40, 44, 48, 52, 56, 60, 64, 68, 72, 76, or 80 with one with one or two conservative amino acid changes.

In certain embodiments, provided herein are methods of treating or preventing a T-cell related disease or condition comprising: treating a subject with a composition comprising a human Cluster of Differentiation 6 (CD6) binding molecule, or an expression vector comprising the one or more nucleic acid molecules encoding said CD6 binding molecule, as recited above and herein, and wherein the subject has, or is suspected to develop, a T-cell related disease or condition. In some embodiments, the T-cell related disease comprises cancer, and optionally wherein the cancer comprises T-cell lymphoma. In other embodiments, the T-cell related disease comprises acute respiratory distress syndrome (ARDS), cytokine-release syndrome in a Covid-19 subject, or acute graft vs host disease (aCGDH). In further embodiments, the T-cell related disease comprises lupus nephritis, uncontrolled asthma, psoriasis, or multiple schlerosis. In particular embodiments, the human CD6 binding molecule is conjugated to a cytotoxic agent, and optionally wherein the cytotoxic agent comprises Monomethyl auristatin (MMAE).

In other embodiments, provided herein are methods of detecting human Cluster of Differentiation 6 (CD6) in a sample comprising: a) contacting a sample with the human CD6 binding molecule as described above and herein, wherein the sample is suspected of containing human CD6, and wherein the human CD6 binding molecule forms a complex with the human CD6 if present in the sample; and b) detecting the presence or absence of the complex in the sample. In some embodiments, the sample is from a subject that has, or is suspected to develop, a T-cell related disease or condition. In other embodiments, the human CD6 binding molecule comprises a detectable label. In additional embodiments, the methods further comprise contacting the sample with a conjugate molecule capable of binding to the human CD6 binding molecule, wherein the conjugate molecule comprises a detectable label.

In certain embodiments, the first SMVAD further comprises four Framework regions, wherein the four Framework regions are camelid, humanized, or human Framework regions.

In additional embodiments, the human CD6 binding molecule further comprises a second SMVAD that comprises: D) a CDR1 amino acid sequence comprising SEQ ID NO:2, 6, 10, 14, 18, 22, 26, 30, 34, 38, 42, 46, 50, 54, 58, 62, 66, 70, 74, or 78; or SEQ ID NO:2, 6, 10, 14, 18, 22, 26, 30, 34, 38, 42, 46, 50, 54, 58, 62, 66, 70, 74, or 78; with one with one or two conservative amino acid changes, E) a CDR2 amino acid sequence comprising SEQ ID NO:3, 7, 11, 15, 19, 23, 27, 31, 35, 39, 43, 47, 51, 55, 59, 63, 67, 71, 75, or 79; or SEQ ID NO:3, 7, 11, 15, 19, 23, 27, 31, 35, 39, 43, 47, 51, 55, 59, 63, 67, 71, 75, or 79 with one or two conservative amino acid changes, and F) a CDR3 amino acid sequence comprising SEQ ID NO:4, 8, 12, 16, 20, 24, 28, 32, 36, 40, 44, 48, 52, 56, 60, 64, 68, 72, 76, or 80; or SEQ ID NO:4, 8, 12, 16, 20, 24, 28, 32, 36, 40, 44, 48, 52, 56, 60, 64, 68, 72, 76, or 80 with one with one or two conservative amino acid changes.

In additional embodiments, the human CD6 binding molecule further comprises a linker which is attached to both the first SMVAD and the second SMVAD. In other embodiments, the one or more nucleic acid molecules comprise: i) a first nucleic acid sequence encoding the first SMVAD, and optionally further encoding a CH2 heavy chain constant region (e.g., which is human or humanized) and/or a CH3 heavy chain constant region (e.g., which is human or humanized) and ii) a second nucleic acid sequence encoding the second SMVAD, and optionally further encoding a CH2 heavy chain constant region (e.g., which is human or humanized) and/or a CH3 heavy chain constant region (e.g., which is human or humanized). In some embodiments, the first SMVAD comprises the amino acid sequence shown in SEQ ID NO:1, 5, 9, 13, 17, 21, 25, 29, 33, 37, 41, 45, 49, 53, 57, 61, 65, 69, 73, or 77; or SEQ ID NO:1, 5, 9, 13, 17, 21, 25, 29, 33, 37, 41, 45, 49, 53, 57, 61, 65, 69, 73, or 77 with one, two, three, or four deletions and/or conservative amino acid changes at either, or both, ends. In other embodiments, the human CD6 binding molecule further comprises a CH2 heavy chain constant region and/or a CH3 heavy chain constant region. In some embodiments, the CH2 and/or CH3 heavy chain constant regions are camelid, humanized, or human. In further embodiments, the human CD6 binding molecule comprises at least an antigen binding portion of Clone 2G1 CD6 nanobody.

In some embodiments, the compositions, kits, and systems herein further comprise a physiologically tolerable buffer. In certain embodiments, the compositions herein comprise the one or more nucleic acid molecules (e.g., first and second nucleic acid molecules), and optionally the composition further comprises an expression vector, and wherein the one or more nucleic acid sequences are present in the expression vector. In particular embodiments, the composition comprises the human CD6 binding molecule. In some embodiments, wherein: the CDR1 amino acid sequence comprises SEQ ID NO:2, 6, 10, 14, 18, 22, 26, 30, 34, 38, 42, 46, 50, 54, 58, 62, 66, 70, 74, or 78; the CDR2 amino acid sequence comprises SEQ ID NO:3, 7, 11, 15, 19, 23, 27, 31, 35, 39, 43, 47, 51, 55, 59, 63, 67, 71, 75, or 79, and the CDR3 amino acid sequence comprises SEQ ID NO:4, 8, 12, 16, 20, 24, 28, 32, 36, 40, 44, 48, 52, 56, 60, 64, 68, 72, 76, or 80.

### DESCRIPTION OF THE FIGURES

FIG. 1 shows the amino acid sequence (SEQ ID NO:1) of Clone 2G1 CD6 nanobody VHH sequence, including CDR1 (SEQ ID NO:2), CDR2 (SEQ ID NO:3), and CDR3 (SEQ ID NO:4).

FIG. 2 shows an exemplary step by step procedure that is used to generate CD6 nanobodies.

FIG. 3 shows that CD6 purified clone 2G1 binds to human CD6 with similar affinity and is detectable at 0.1pM.

FIG. 4 shows clone 2G1 nanobody binds to a T cell line (CD6+) dose-dependently.

FIG. 5 shows an exemplary design of a next generation CD6-ADC based novel bivalent anti-CD6 nanobody.

FIGS. 6A and 6B show that engineered CD6 nanobodies selectively bind to CD6 on T cells.

FIG. 7 shows that bivalent CD6 nanobody is internalized by T cells (HuT 78).

FIG. 8 shows that next generation CD6-ADC kills T cell lymphoma cells better than the first generation CD6-ADC.

FIG. 9 shows that CD6 nanobody dimer (2G1-2G1) linked MMAE (Monomethyl auristatin) inhibits human T cell (HH cell line) proliferation.

FIG. 10A shows the amino acid sequence (SEQ ID NO:5) of Clone A1CD601 chimeric nanobody VHH sequence, including CDR1 (SEQ ID NO:6), CDR2 (SEQ ID NO:7), and CDR3 (SEQ ID NO:8). FIG. 10B shows the amino acid sequence (SEQ ID NO:9) of Clone CD601HH1 nanobody VHH sequence, including CDR1 (SEQ ID NO:10), CDR2 (SEQ ID NO:11), and CDR3 (SEQ ID NO:12). FIG. 10C shows the amino acid sequence (SEQ ID NO:13) of Clone CD601HH2 nanobody VHH sequence, including CDR1 (SEQ ID NO:14), CDR2 (SEQ ID NO:15), and CDR3 (SEQ ID NO:16). FIG. 10D shows the amino acid sequence (SEQ ID NO:17) of Clone CD601HH3 nanobody VHH sequence, including CDR1 (SEQ ID NO:18), CDR2 (SEQ ID NO:19), and CDR3 (SEQ ID NO:20).

FIG. 11A shows the amino acid sequence (SEQ ID NO:21) of Clone CD601HH4 chimeric nanobody VHH sequence, including CDR1 (SEQ ID NO:22), CDR2 (SEQ ID NO:23), and CDR3 (SEQ ID NO:24). FIG. 11B shows the amino acid sequence (SEQ ID NO:25) of Clone CD601HH5 nanobody VHH sequence, including CDR1 (SEQ ID NO:26), CDR2 (SEQ ID NO:27), and CDR3 (SEQ ID NO:28). FIG. 11C shows the amino acid sequence (SEQ ID NO:29) of Clone CD601HH6 nanobody VHH sequence, including CDR1 (SEQ ID NO:30), CDR2 (SEQ ID NO:31), and CDR3 (SEQ ID NO:32). FIG. 11D shows the amino acid sequence (SEQ ID NO:33) of Clone CD601HH7 nanobody VHH sequence, including CDR1 (SEQ ID NO:34), CDR2 (SEQ ID NO:35), and CDR3 (SEQ ID NO:36).

FIG. 12A shows the amino acid sequence (SEQ ID NO:37) of Clone CD601HH8 chimeric nanobody VHH sequence, including CDR1 (SEQ ID NO:38), CDR2 (SEQ ID NO:39), and CDR3 (SEQ ID NO:40). FIG. 12B shows the amino acid sequence (SEQ ID NO:41) of Clone CD601HH9 nanobody VHH sequence, including CDR1 (SEQ ID NO:42), CDR2 (SEQ ID NO:43), and CDR3 (SEQ ID NO:44). FIG. 12C shows the amino acid sequence (SEQ ID NO:45) of Clone CD601HH10 nanobody VHH sequence, including CDR1 (SEQ ID NO:46), CDR2 (SEQ ID NO:47), and CDR3 (SEQ ID NO:48). FIG. 12D shows the amino acid sequence (SEQ ID NO:49) of Clone CD601HH11 nanobody VHH sequence, including CDR1 (SEQ ID NO:50), CDR2 (SEQ ID NO:51), and CDR3 (SEQ ID NO:52).

FIG. 13A shows the amino acid sequence (SEQ ID NO:53) of Clone CD601HH12 chimeric nanobody VHH sequence, including CDR1 (SEQ ID NO:54), CDR2 (SEQ ID NO:55), and CDR3 (SEQ ID NO:56). FIG. 13B shows the amino acid sequence (SEQ ID NO:157) of Clone CD601HH13 nanobody VHH sequence, including CDR1 (SEQ ID NO:58), CDR2 (SEQ ID NO:59), and CDR3 (SEQ ID NO:60). FIG. 13C shows the amino acid sequence (SEQ ID NO:61) of Clone CD601HH14 nanobody VHH sequence, including CDR1 (SEQ ID NO:62), CDR2 (SEQ ID NO:63), and CDR3 (SEQ ID NO:64). FIG. 13D shows the amino acid sequence (SEQ ID NO:65) of Clone CD601HH15 nanobody VHH sequence, including CDR1 (SEQ ID NO:66), CDR2 (SEQ ID NO:67), and CDR3 (SEQ ID NO:68).

FIG. 14A shows the amino acid sequence (SEQ ID NO:69) of Clone CD601HH16 chimeric nanobody VHH sequence, including CDR1 (SEQ ID NO:70), CDR2 (SEQ ID NO:71), and CDR3 (SEQ ID NO:72). FIG. 14B shows the amino acid sequence (SEQ ID NO:73) of Clone CD601HH17 nanobody VHH sequence, including CDR1 (SEQ ID NO:74), CDR2 (SEQ ID NO:75), and CDR3 (SEQ ID NO:76). FIG. 14C shows the amino acid sequence (SEQ ID NO:77) of Clone CD601HH18 nanobody VHH sequence, including CDR1 (SEQ ID NO:78), CDR2 (SEQ ID NO:79), and CDR3 (SEQ ID NO:80).

FIG. 15 shows results of a CD6-ADC assay based on the humanized CD6 nanobody kills T cell lymphoma cells. One of the humanized CD6 nanobody clones (HH4) was conjugated with MMAE to develop the next generation of CD6-ADC. T cell lymphoma cell line HH cells were cultured with 0-128 nM of the new CD6-ADC (HH14-MMAE) or a control (hIgG-MMAE) for 72 hrs, and the cell killing was quantitated using trypan blue to distinguish dead from live cells.

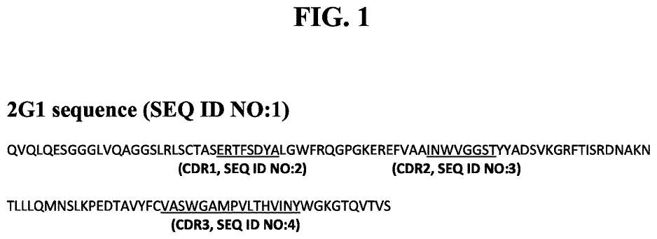

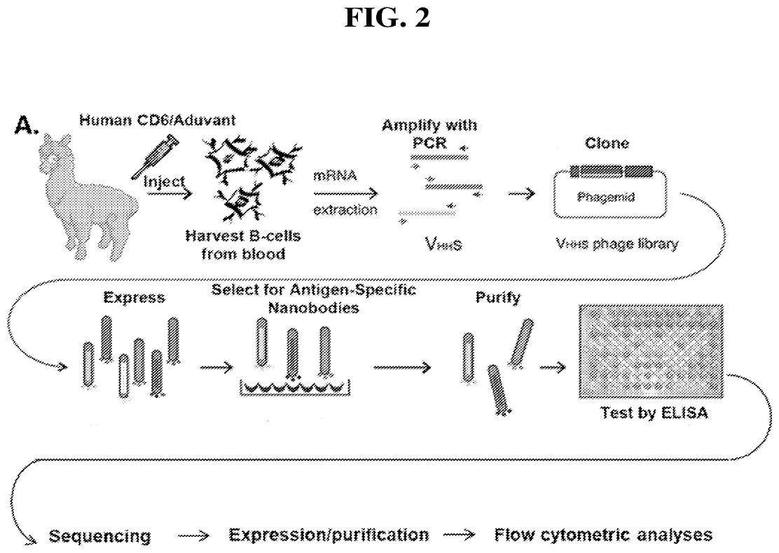

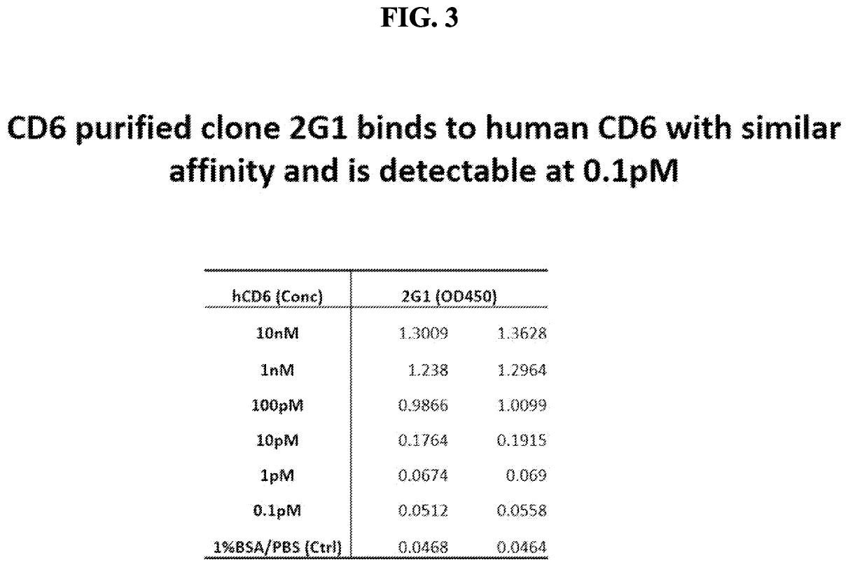

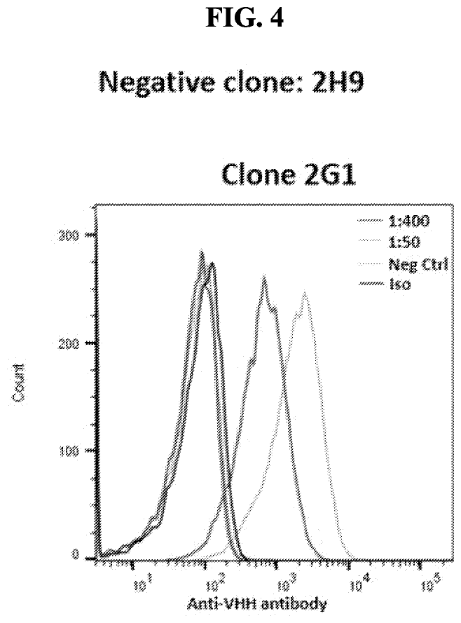

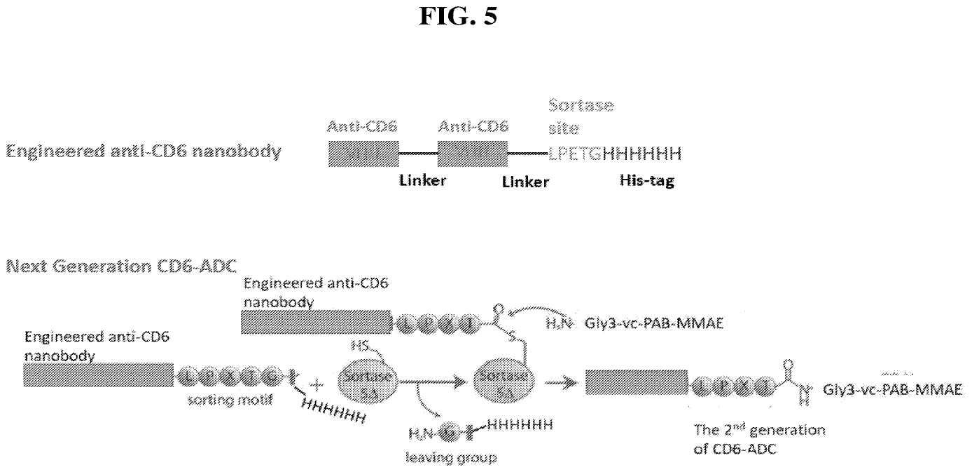

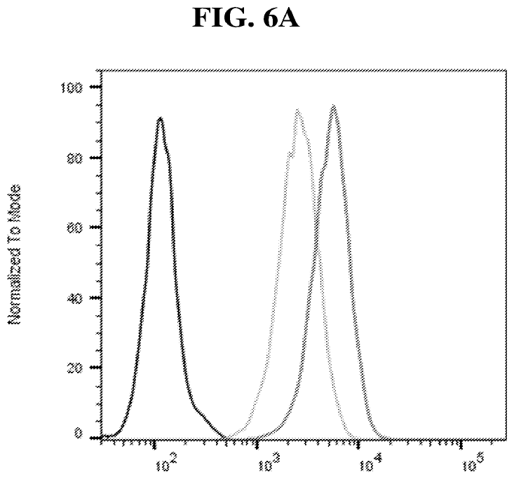

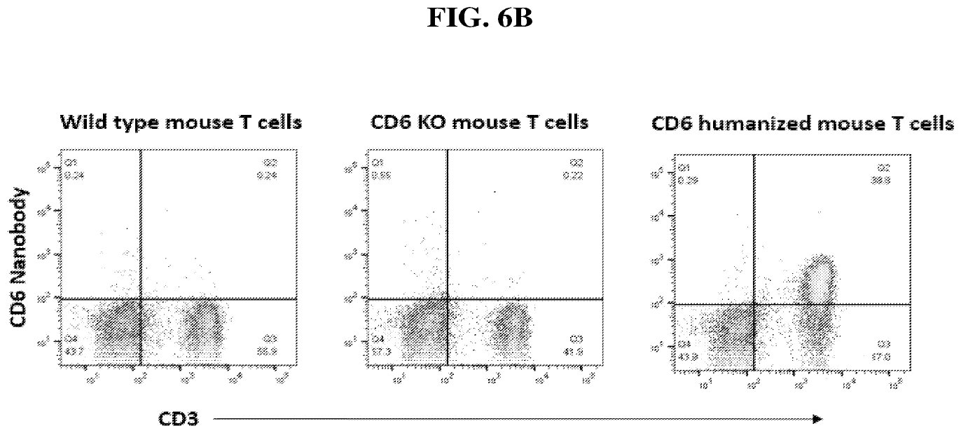

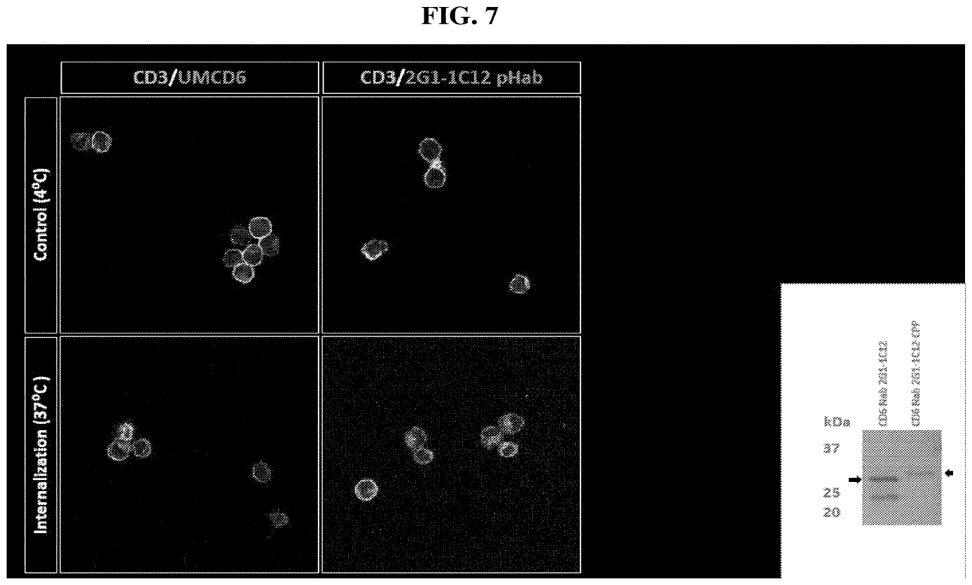

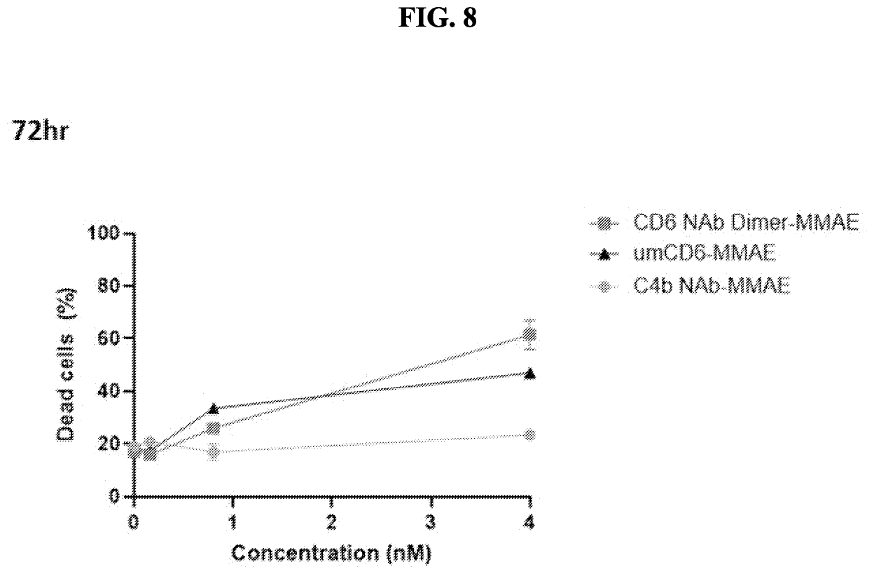

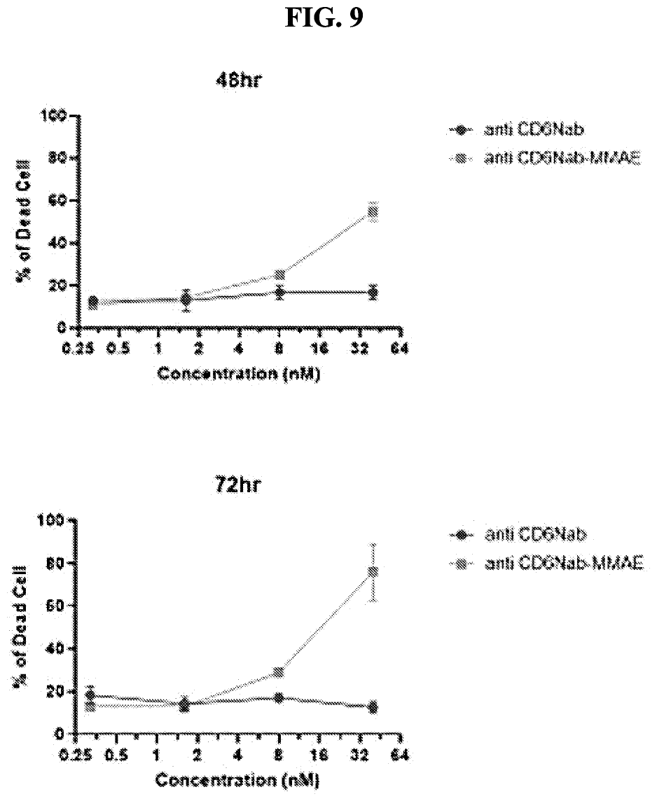

### DEFINITIONS

To facilitate an understanding of the invention, a number of terms are defined below.

A “nanobody,” or “single variable domain” (“VHH”) or “single monomeric variable antibody domain” (“SMVAD”) as used herein, refer to the smallest antigen binding fragment originally derived from a naturally occurring heavy chain antibody and is known to the person skilled in the art. Such nanobodies can be derived from antibodies raised in Camelidae species, for example in camel, llama, dromedary, alpaca and guanaco. Nanobodies may also be synthetically produced, such as by overexpression in bacteria. Single domain antibodies are antibodies whose complementary determining regions (CDRs) are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies.

As used herein, the terms “subject” and “patient” refer to any animal, such as a mammal like a dog, cat, bird, livestock, and preferably a human.

As used herein, the term “codon” or “triplet” refers to a group of three adjacent nucleotides which specify one of the naturally occurring amino acids found in polypeptides. The term also includes codons which do not specify any amino acid. It is also noted that, due to the degeneracy of the genetic code, there are many codons that code for the same amino acid. As such, many of the bases of the nucleic acid sequences of the present invention can be changed without changing the actual amino acid sequence that is encoded. The present disclosure is intended to encompass all such nucleic acid sequences.

As used herein, the terms “an oligonucleotide having a nucleotide sequence encoding a polypeptide,” “polynucleotide having a nucleotide sequence encoding a polypeptide,” and “nucleic acid sequence encoding a peptide” means a nucleic acid sequence comprising the coding region of a particular polypeptide. The coding region may be, for example, present in a cDNA, genomic DNA, or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc., or a combination of both endogenous and exogenous control elements.

The term “isolated” when used in relation to a nucleic acid, as in “an isolated oligonucleotide” or “isolated polynucleotide” or “isolated nucleic acid sequence encoding a Cluster of Differentiation 6 binding molecule” refers to a nucleic acid sequence that is identified and separated from at least one contaminant nucleic acid with which it is ordinarily associated (e.g. host cell proteins).

As used herein, the term “purified” or “to purify” refers to the removal of contaminants from a sample. For example, Cluster of Differentiation 6 binding molecules may be purified by removal of contaminating non-immunoglobulin proteins; they are also purified by the removal of immunoglobulins that do not bind to the same antigen. The removal of non-immunoglobulin proteins and/or the removal of immunoglobulins that do not bind the particular antigen results in an increase in the percentage of antigen specific immunoglobulins in the sample. In another example, recombinant antigen-specific polypeptides are expressed in bacterial host cells and the polypeptides are purified by the removal of host cell proteins; the percentage of recombinant antigen-specific polypeptides is thereby increased in the sample.

### DESCRIPTION OF THE INVENTION

Provided herein are human Cluster of Differentiation 6 (CD6) binding molecules and nucleic acid sequences encoding such molecules. In particular embodiments, provided herein are human CD6 binding molecules (e.g., nanobodies) having a first, and optionally a second, single monomeric variable antibody domain (SMVAD) (aka a “nanobody”) that comprises certain CDRs, and methods for using such molecules to treat T-cell related diseases (e.g., cancer, such as T-cell lymphoma). In certain embodiments, the SMVAD comprises camelid, human, or humanized framework regions.

The nanobodies (SMVADs), according to the present disclosure, in certain embodiments, generally comprise a single amino acid chain that can be considered to comprise 4 “framework sequences” or FRs and 2 or 3 “complementary determining regions” or CDRs, preferably in a sequence FR1-CDR1-FR2-CDR2-FR3-(optionally CDR3)-FR4. Non-limiting examples of nanobodies of the disclosure are described in more detail further herein. It should be clear that framework regions of nanobodies may also contribute to the binding of their antigens. It should however be noted that parts, fragments, analogs or derivatives (as further described herein) of a nanobody are not particularly limited as to their length and/or size, as long as such parts, fragments, analogs or derivatives meet the further requirements outlined herein and are also preferably suitable for the purposes described herein.

The terms “nanobody” and “SMVAD,” in their broadest sense, are not limited to a specific biological source or to a specific method of preparation. For example, the nanobodies of the disclosure can generally be obtained: (1) by isolating the VHH domain of a naturally occurring heavy chain antibody; (2) by expression of a nucleotide sequence encoding a naturally occurring VHH domain; (3) by “humanization” of a naturally occurring VHH domain or by expression of a nucleic acid encoding a such humanized VHH domain (see, e.g., Sulea, Humanization of Camelid Single Domain Antibodies, Methods Mol Biol. 2022; 2446:299-312 and Vincke et al., General Strategy to Humanize a Camelid Single-domain Antibody and Identification of a Universal Humanized Nanobody Scaffold, The J. of Bio. Chem. Vol. 284, No. 5, pp. 3273-3284, Jan. 30, 2009; both of which are herein incorporated in their entireties and particularly for methods of humanizing nanobodies); (4) by “camelization” of a naturally occurring VH domain from any animal species, and in particular from a mammalian species, such as from a human being, or by expression of a nucleic acid encoding such a camelized VH domain; (5) by “camelization” of a “domain antibody” or “Dab,” as described in the art, or by expression of a nucleic acid encoding such a camelized VH domain; (6) by using synthetic or semi-synthetic techniques for preparing proteins, polypeptides or other amino acid sequences known per se; (7) by preparing a nucleic acid encoding a nanobody using techniques for nucleic acid synthesis known per se, followed by expression of the nucleic acid thus obtained; and/or (8) by any combination of one or more of the foregoing.

The small size and unique biophysical properties of nanobodies generally exceed conventional antibody fragments for the recognition of uncommon or hidden epitopes and for binding into cavities or active sites of protein targets. Further, nanobodies herein can be designed as bispecific and bivalent antibodies or attached to reporter molecules. Nanobodies are stable and rigid single domain proteins that can generally be easily be manufactured and survive the gastro-intestinal system.

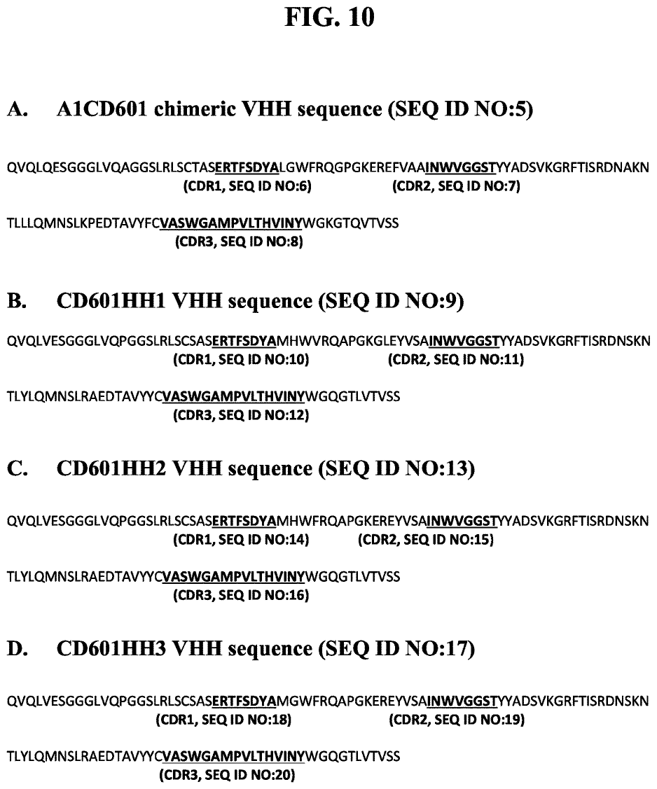

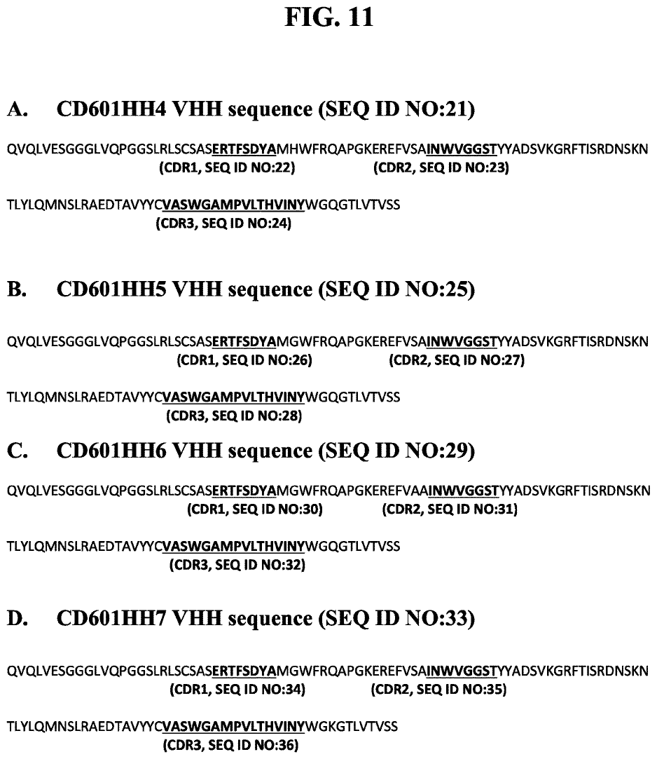

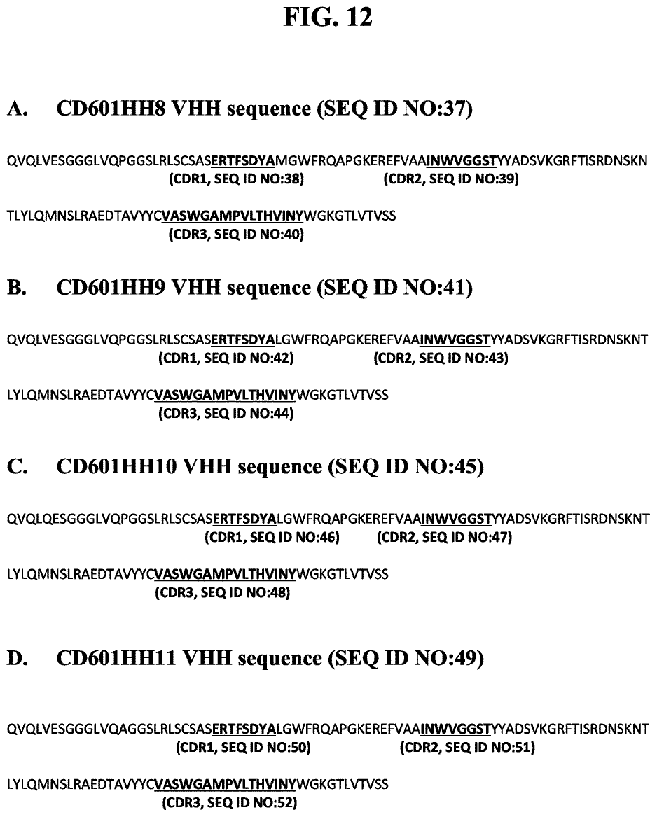

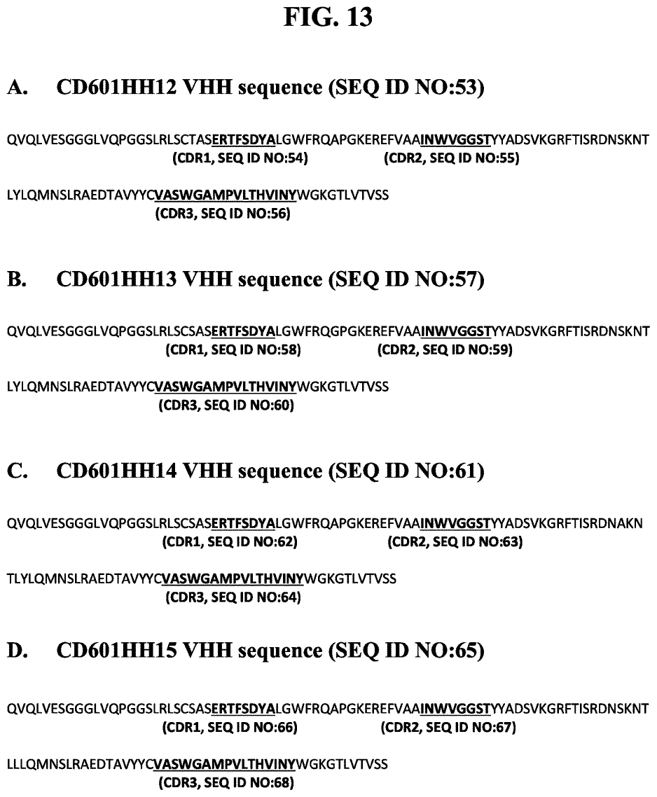

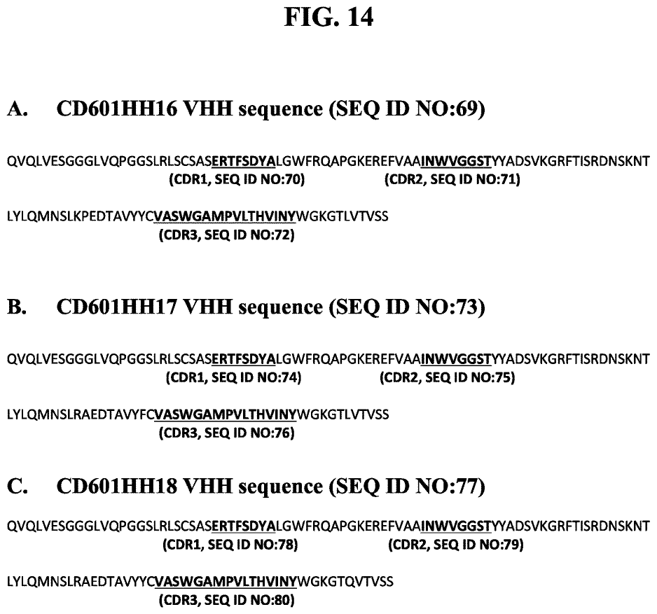

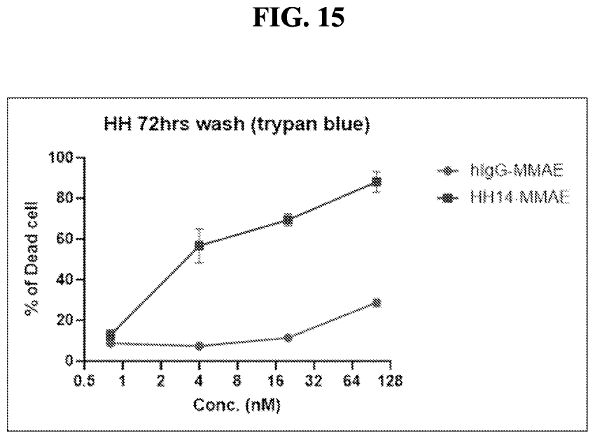

Work conducted during development of embodiments herein developed the VHH sequences for a chimeric antibody (A1CD601 VHH) and 18 humanized antibodies (CD601HH1-18 VHH). The sequences of the VHH sequences are shown in FIG. 10-14, with an alignment shown in FIG. 15. The properties of four of these antibodies (CD601HH 8, 10, 12, and 14 HuG1Fc), as well as the chimeric antibody (A1CD601 HuG1Fc) are shown in Table 1 below. The sequence employed for HuG1Fc expression for these antibodies (SEQ ID NO:83) is shown in Table 2 below.

TABLE 1

| | Chimeric | CD601 Humanized candidates | | | |
|---|---|---|---|---|---|
| | AlCD601VHH | CD601HH8 | CD601HH10 | CD601HH12 | CD601HH14 |
| Property | HuG1Fc | HuG1Fc | HuG1Fc | HuG1Fc | HuG1Fc |
| CD6 binding by ELISA | 0.053 | 0.053 | 0.066 | 0.050 | 0.053 |
| (EC₅₀, nM) | | | | | |
| Selectivity (no Hu-CD5 | Selective | Selective | Selective | Selective | Selective |
| binding by ELISA) | | | | | |
| Cross-reactivity (binding | Cross-reactive | Cross-reactive | Cross-reactive | Cross-reactive | Cross- |
| to cyno-CD6 by ELISA) | | | | | reactive |
| KD (M) | 2.10 × 10⁻¹⁰ | 2.40 × 10⁻¹⁰ | 2.47 × 10⁻¹⁰ | 2.22 × 10⁻¹⁰ | 2.69 × 10⁻¹⁰ |
| Flow data: Geometric | 94.3 | 194 | 148 | 158 | 196 |
| mean at 4 ng/ml (CC) | | | | | |
| pI range of all isoforms | 6.88-8.34 | 6.91-8.20 | 6.88-8.20 | 6.94-8.26 | 6.93-8.21 |
| (PA800) | | | | | |
| pI range of all isoforms | 7.47-8.86 | 7.51-8.85 | 7.51-8.86 | 7.51-8.85 | 7.48-8.85 |
| (Maurice) | | | | | |
| Tₘ (° C.) | 61.8 | 66.9 | 65.8 | 67.7 | 66.2 |
| T_{agg} (° C.) | 72.6 | 70.9 | 73.9 | 73.5 | 72.6 |
| Minimum solubility - pH | — | — | — | 52 | 72 |
| 6.0 (mg/ml) | | | | | |
| 1 month 50° C. heat stress | 59% | 12.5% | 14.6% | 5.7% | 8.9% |
| (aggregation by SEC- | | | | | |
| MALS) | | | | | |
| Freeze-thaw stress | 3.9% | 1.2% | 2.3% | 1.7% | 5.1% |
| (aggregation by SEC- | | | | | |
| MALS) | | | | | |
| Non-specific interactions | 0.019 | 0.025 | 0.045 | 0.025 | 0.015 |
| (k′) | | | | | |
| Serum stability (human, | Retained | Retained | Retained | Retained | Retained |
| cyno and mouse) | binding | binding | binding | binding | binding |
| Thermal stability (50% | 64.0 | 67.5 | 66.5 | 68.0 | 67.0 |
| loss of binding temp, ° C.) | | | | | |
| VHH: % ID with closest | 77.1% | 87.5% | 85.4% | 85.4% | 85.4% |
| human germline V- | (IGHV3-64*03) | (IGHV3-64*04) | (IGHV3-64*04) | (IGHV3-64*04) | (IGHV3-64*04) |
| segment | | | | | |

TABLE 2
Amino acid sequences of signal peptide and constant regions

| Name | Sequence |
|---|---|
| Human signal | MEAPAQLLFLLLLWLPDTTG (SEQ ID NO: 81) |
| peptide * | |
| Human IgG1 | EPKSSDKTHTCPPCPAPELLGGPSVFLFPPKPKDTLMISRTPEVT |
| hinge, CH2 and | CVVVDVSHEDPEVKFNWYVDGVEVHNAKTKPREEQYNSTYRVVSV |
| CH3 | LTVLHQDWLNGKEYKCKVSNKALPAPIEKTISKAKGQPREPQVYT |
| | LPPSREEMTKNQVSLTCLVKGFYPSDIAVEWESNGQPENNYKTTP |
| | PVLDSDGSFFLYSKLTVDKSRWQQGNVFSCSVMHEALHNHYTQKS |
| | LSLSPGK (SEQ ID NO: 82) |
| Complete human | ASTKGPSVFPLAPSSKSTSGGTAALGCLVKDYFPEPVTVSWNSGA |
| IgG1 constant | LTSGVHTFPAVLQSSGLYSLSSVVTVPSSSLGTQTYICNVNHKPS |
| region (CH1, | NTKVDKKVEPKSCDKTHTCPPCPAPELLGGPSVFLFPPKPKDTLM |
| hinge, CH2 and | ISRTPEVTCVVVDVSHEDPEVKFNWYVDGVEVHNAKTKPREEQYN |
| CH3) | STYRVVSVLTVLHQDWLNGKEYKCKVSNKALPAPIEKTISKAKGQ |
| | PREPQVYTLPPSREEMTKNQVSLTCLVKGFYPSDIAVEWESNGQP |
| | ENNYKTTPPVLDSDGSFFLYSKLTVDKSRWQQGNVFSCSVMHEAL |
| | HNHYTQKSLSLSPGK (SEQ ID NO: 83) |
| Human kappa | RTVAAPSVFIFPPSDEQLKSGTASVVCLLNNFYPREAKVQWKVDN |
| constant region | ALQSGNSQESVTEQDSKDSTYSLSSTLTLSKADYEKHKVYACEVT |
| | HQGLSSPVTKSFNRGEC (SEQ ID NO: 84) |
| VHH tag (6H_C) | HHHHHHC (SEQ ID NO: 85) |

Other constant regions known in the art may be employed with the VHHs herein than those listed in Table 3, which are only examples.

The amino acid residues of a nanobody are generally numbered according to the general numbering for VH domains given by Kabat et al., as applied to VHH domains from Camelids in the article of Riechmann and Muyldermans, J Immunol Methods 1999 Dec. 10;231(1-2):25-38, herein incorporated by reference. According to this numbering, FR1 of a Nanobody comprises the amino acid residues at positions 1-30, CDR1 of a Nanobody comprises the amino acid residues at positions 31-35, FR2 of a Nanobody comprises the amino acids at positions 36-49, CDR2 of a Nanobody comprises the amino acid residues at positions 50-65, FR3 of a Nanobody comprises the amino acid residues at positions 66-94, CDR3 of a Nanobody comprises the amino acid residues at positions 95-102, and FR4 of a Nanobody comprises the amino acid residues at positions 103-1.13. It should be noted that it is well known in the art for VH domains and for VHH domains that the total number of amino acid residues in each of the CDR's may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. Generally, however, it can be said that, according to the numbering of Kabat and irrespective of the number of amino acid residues in the CDR's, position 1 according to the Kabat numbering corresponds to the start of FR1 and vice versa, position 36 according to the Kabat numbering corresponds to the start of FR2 and vice versa, position 66 according to the Kabat numbering corresponds to the start of FR3 and vice versa, and position 103 according to the Kabat numbering corresponds to the start of FR4 and vice versa.

Nanobodies have a number of unique structural characteristics and functional properties which make isolated SMVADs, and proteins containing the same, highly advantageous for use as functional antigen-binding domains or proteins. In particular, and without being limited thereto, SMVADs, which have been “designed” by nature to functionally bind to an antigen without the presence of, and without any interaction with, a light chain variable domain, can function as a single, relatively small, functional antigen-binding structural unit, domain or protein. This distinguishes the nanobodies from the VH and VL domains of conventional 4-chain antibodies, which by themselves are generally not suited for practical application as single antigen-binding proteins or domains, but need to be combined in some form or another to provide a functional antigen-binding unit (as in for example conventional antibody fragments such as Fab fragments; in ScFv's fragments, which are composed of a VH domain covalently linked to a VL, domain).

In certain embodiments, the SMVADs (nanobodies) may be further modified by one or more other amino substitutions while maintaining their activity as CD6 binding molecules. In certain embodiments, substitutions are made in the framework regions and not in the CDR domains. For example, amino acid substitutions can be made at one or more positions wherein the substitution is for an amino acid having a similar hydrophilicity. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules. Thus such conservative substitution can be made in a SMVADs of the embodiments and will likely only have minor effects on their activity. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (0.5); histidine −0.5); cysteine (−1.0); methionine (−1.3); valine (−1.5): leucine (−1.8); isoleucine (−1.8); tyrosine (−2.3); phenylalanine (−2.5); tryptophan (−3.4). These values can be used as a guide and thus substitution of amino acids whose hydrophilicity values are within 2 are preferred, those that are within 1 are particularly preferred, and those within 0.5 are even more particularly preferred. Thus, any of the SMVADs described herein may be modified by the substitution of an amino acid, for different, but homologous amino acid with a similar hydrophilicity value. Amino acids with hydrophilicities within +/−1.0, or +/−0.5 points are considered homologous. Furthermore, it is envisioned that SMVAD sequences may be modified by amino acid deletions, substitutions, additions or insertions while retaining its binding activity.

In certain embodiments, the human Cluster of Differentiation 6 binding molecules comprise one or more of the CDRs shown in SEQ ID NOS: 2-4, 6-8, 10-12, 14-16, 18-20, 22-24, 26-28, 30-32, 34-36, 38-40, 42-44, 46-48, 50-52, 54-56, 58-60, 62-64, 66-68, 70-72, 74-76, 78-80, and/or variable region or CDRs with one or more conservative or non-conservative amino acid changes in these SEQ ID NOS:1-80, and nucleic acid sequences encoding SEQ ID NOs:1-80. Changes to the amino acid sequences of the CDRs or variable regions may be generated by changing the nucleic acid sequence encoding the amino acid sequence. A nucleic acid sequence encoding a variant of a given CDR or variable region may be prepared by methods known in the art using the guidance of the present specification for particular sequences. These methods include, but are not limited to, preparation by site-directed (or oligonucleotide-mediated) mutagenesis, PCR mutagenesis, and cassette mutagenesis of an earlier prepared nucleic acid encoding the CDR or variable region.

Briefly, in carrying out site-directed mutagenesis of DNA, the starting DNA is altered by first hybridizing an oligonucleotide encoding the desired mutation to a single strand of such starting DNA. After hybridization, a DNA polymerase is used to synthesize an entire second strand, using the hybridized oligonucleotide as a primer, and using the single strand of the starting DNA as a template. Thus, the oligonucleotide encoding the desired mutation is incorporated in the resulting double-stranded DNA.

PCR mutagenesis is also suitable for making amino acid sequence variants of the starting CDR (see, e.g., Vallette et. al., (1989) Nucleic Acids Res. 17: 723-733, hereby incorporated by reference). Briefly, when small amounts of template DNA are used as starting material in a PCR, primers that differ slightly in sequence from the corresponding region in a template DNA can be used to generate relatively large quantities of a specific DNA fragment that differs from the template sequence only at the positions where the primers differ from the template.

Another method for preparing variants, cassette mutagenesis, is based on the technique described by Wells et al., (1985) Gene 34: 315-323, hereby incorporated by reference. The starting material is the plasmid (or other vector) comprising the starting CDR or variant region DNA to be mutated. The codon(s) in the starting DNA to be mutated are identified. There should be a unique restriction endonuclease site on each side of the identified mutation site(s). If no such restriction sites exist, they may be generated using the above-described oligonucleotide-mediated mutagenesis method to introduce them at appropriate locations in the starting polypeptide DNA. The plasmid DNA is cut at these sites to linearize it. A double-stranded oligonucleotide encoding the sequence of the DNA between the restriction sites but containing the desired mutation(s) is synthesized using standard procedures, wherein the two strands of the oligonucleotide are synthesized separately and then hybridized together using standard techniques. This double-stranded oligonucleotide is referred to as the cassette. This cassette is designed to have 5′ and 3′ ends that are compatible with the ends of the linearized plasmid, such that it can be directly ligated to the plasmid. This plasmid now contains the mutated DNA sequence.

Alternatively, or additionally, the desired amino acid sequence encoding a CDR variant, or variable region variant, can be determined, and a nucleic acid sequence encoding such amino acid sequence variant can be generated synthetically. Conservative modifications in the amino acid sequences of the CDRs or variable region may also be made. Naturally occurring residues are divided into classes based on common side-chain properties:
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic: trp, tyr, phe.
   Conservative substitutions will entail exchanging a member of one of these classes for another member of the same class in a particular antibody, variable region, or CDR, such as in SEQ ID NOS: 1-80.

For expression of human CD6 binding molecules disclosed herein, the expression vector(s) encoding the SMVADs may be transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like.

In certain embodiments, the expression vector used to express the human CD6 binding molecules of the present invention are viral vectors, such as retro-viral vectors. Such viral vectors may be employed to generate stably transduced cell lines (e.g. for a continues source of the Cluster of Differentiation 6 binding molecules). In some embodiments, the GPEX gene product expression technology (from Catalent, Somerset, NJ) is employed to generate Cluster of Differentiation 6 binding molecules (and stable cell lines expressing the Cluster of Differentiation 6 binding molecules). In particular embodiments, the expression technology described in WO0202783 and WO0202738 (both of which are herein incorporated by reference in their entireties) is employed.

Mammalian host cells for expressing the human CD6 binding molecules of the invention include, for example, PER.Cluster of Differentiation 6™ cells (Crucell, The Netherlands), Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NSO myeloma cells, COS cells and SP2 cells. When recombinant expression vectors encoding the human CD6 binding molecules are introduced into mammalian host cells, the antibodies are generally produced by culturing the host cells for a period of time sufficient to allow for expression of the human CD6 binding molecules in the host cells or, more preferably, secretion of the nanobody into the culture medium in which the host cells are grown. Human CD6 binding molecules can be recovered from the culture medium using standard protein purification methods.

In certain embodiments, the human Cluster of Differentiation 6 binding molecules of the present invention (e.g., nanobodies or dual nanobodies) are useful for immunoassays which detect or quantify human Cluster of Differentiation 6 in a sample (e.g., a purified blood sample from a subject). In some embodiments, an immunoassay for Cluster of Differentiation 6 typically comprises incubating a biological sample in the presence of a detectably labeled antibody or antibody fragment of the present invention capable of selectively binding to Cluster of Differentiation 6, and detecting the labeled peptide or antibody which is bound in a sample. Various clinical assay procedures are well known in the art.

The present disclosure provides immunoassay methods for determining the presence, amount or concentration of human Cluster of Differentiation 6 in a test sample. Any suitable assay known in the art can be used in such a method. Examples of such assays include, but are not limited to, immunoassay, such as sandwich immunoassay (e.g., monoclonal-polyclonal sandwich immunoassays, including radioisotope detection (radioimmunoassay (RIA)) and enzyme detection (enzyme immunoassay (EIA) or enzyme-linked immunosorbent assay (ELISA) (e.g., Quantikine ELISA assays, R&D Systems, Minneapolis, Minn.)), competitive inhibition immunoassay (e.g., forward and reverse), fluorescence polarization immunoassay (FPIA), enzyme multiplied immunoassay technique (EMIT), an ARCHITECT assay (ABBOTT), a bioluminescence resonance energy transfer (BRET), and homogeneous chemiluminescent assay, etc.

A human CD6 binding molecule can be captured on beads or nitrocellulose, or on any other solid support which is capable of immobilizing soluble proteins (e.g., magnetic beads). A human CD6 containing sample is then added to the support which is subsequently washed with suitable buffers to remove unbound proteins. A second, detectably labeled, molecule (e.g., antibody or peptide) that can bind to the human CD6 binding molecule is added to the solid phase support that can then be washed with the buffer a second time to remove unbound molecules. The amount of bound label on the solid support can then be detected by known methods.

Detectably labeling the human CD6 binding molecules can be accomplished by coupling to an enzyme for use in an enzyme immunoassay (EIA), or enzyme-linked immunosorbent assay (ELISA). The linked enzyme reacts with the exposed substrate to generate a chemical moiety which can be detected, for example, by spectrophotometric, fluorometric or by visual means. Enzymes which can be used to detectably label the human CD6 binding molecules of the present invention include, but are not limited to, malate dehydrogenase, staphylococcal nuclease, delta-5-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase.

In some embodiments of the present invention, human CD6 which is detected by the above assays can be present in a biological sample. Any sample containing human CD6 can be used. Preferably, the sample is a biological fluid such as, for example, blood, brain tissue, serum, lymph, urine, cerebrospinal fluid, amniotic fluid, synovial fluid, a tissue extract or homogenate, and the like. However, the invention is not limited to assays using only these samples, as it is possible for one of ordinary skill in the art to determine suitable conditions which allow the use of other samples.

In situ detection can be accomplished by removing a histological specimen from a patient, and providing the combination of labeled human CD6 binding molecules of the present disclosure to such a specimen. The human CD6 binding molecule is preferably provided by applying or by overlaying the labeled CD6 binding molecule to a biological sample (e.g., brain tissue). Through the use of such a procedure, it is possible to determine not only the presence of CD6, but also the distribution of CD6 in the examined tissue.

In certain embodiments, provided here are kits for the detection of Cluster of Differentiation 6 that include a human Cluster of Differentiation 6 detection molecule. Such kits may include any of the immunodiagnostic reagents described herein and may further include instructions for the use of the immunodiagnostic reagents in immunoassays for determining the presence of human Cluster of Differentiation 6 in a test sample. The kits may also include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kit may be provided in suitable containers as necessary, e.g., a microtiter plate. The kit can further include containers for holding or storing a sample (e.g., a container or cartridge for a sample). Where appropriate, the kit optionally also can contain reaction vessels, mixing vessels, and other components that facilitate the preparation of reagents or the test sample. The kit can also include one or more instrument for assisting with obtaining a test sample, such as a syringe, pipette, forceps, measured spoon, or the like.

### REFERENCES

Aleandri, S., et al. Dynamic Light Scattering of Biopharmaceutics-Can Analytical Performance Be Enhanced by Laser Power? Pharmaceutics (2018) 10: 94.
Crauwels, M., et al. Reshaping nanobodies for affinity purification on protein a. N Biotechnol. (2020) 57:20-28.
Garner, L. I., et al., CD6 monoclonal antibodies differ in epitope, kinetics and mechanism of action. Immunology (2018) 155: 273-282.
Nguyen, V. K., et al. Camel heavy-chain antibodies: diverse germline V(H)H and specific mechanisms enlarge the antigen-binding repertoire. EMBO J. (2000) 19: 921-30.
Sydow, J. F., et al. Structure-based prediction of asparagine and aspartate degradation sites in antibody variable regions. PLoS One (2014) 9(6):e100736.
Vincke, C., et al. General strategy to humanize a camelid single-domain antibody and identification of a universal humanized nanobody scaffold. J Biol Chem. (2009) 284: 3273-3284|doi: 10.1074/jbc.M806889200|

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, medicine, and molecular biology or related fields are intended to be within the scope of the following claims.

We claim:

1. A composition comprising a human Cluster of Differentiation 6 (CD6) binding molecule, or one or more nucleic acid molecules encoding said human CD6 binding molecule,
wherein said human CD6 binding molecule comprises a first single monomeric variable antibody domain (SMVAD) that comprises:
A) a CDR1 amino acid sequence comprising SEQ ID NO:2, 6, 10, 14, 18, 22, 26, 30, 34, 38, 42, 46, 50, 54, 58, 62, 66, 70, 74, or 78; or SEQ ID NO:2, 6, 10, 14, 18, 22, 26, 30, 34, 38, 42, 46, 50, 54, 58, 62, 66, 70, 74, or 78; with one with one or two conservative amino acid changes,
B) a CDR2 amino acid sequence comprising SEQ ID NO:3, 7, 11, 15, 19, 23, 27, 31, 35, 39, 43, 47, 51, 55, 59, 63, 67, 71, 75, or 79; or SEQ ID NO:3, 7, 11, 15, 19, 23, 27, 31, 35, 39, 43, 47, 51, 55, 59, 63, 67, 71, 75, or 79 with one or two conservative amino acid changes, and
C) a CDR3 amino acid sequence comprising SEQ ID NO:4, 8, 12, 16, 20, 24, 28, 32, 36, 40, 44, 48, 52, 56, 60, 64, 68, 72, 76, or 80; or SEQ ID NO:4, 8, 12, 16, 20, 24, 28, 32, 36, 40, 44, 48, 52, 56, 60, 64, 68, 72, 76, or 80 with one with one or two conservative amino acid changes.

2. The composition of claim 1, wherein said first SMVAD further comprises four Framework regions, wherein said four Framework regions are camelid, humanized, or human Framework regions.

3. The composition of claim 1, wherein said human CD6 binding molecule further comprises a second SMVAD that comprises:
D) a CDR1 amino acid sequence comprising SEQ ID NO:2, 6, 10, 14, 18, 22, 26, 30, 34, 38, 42, 46, 50, 54, 58, 62, 66, 70, 74, or 78; or SEQ ID NO:2, 6, 10, 14, 18, 22, 26, 30, 34, 38, 42, 46, 50, 54, 58, 62, 66, 70, 74, or 78; with one with one or two conservative amino acid changes,
E) a CDR2 amino acid sequence comprising SEQ ID NO:3, 7, 11, 15, 19, 23, 27, 31, 35, 39, 43, 47, 51, 55, 59, 63, 67, 71, 75, or 79; or SEQ ID NO:3, 7, 11, 15, 19, 23, 27, 31, 35, 39, 43, 47, 51, 55, 59, 63, 67, 71, 75, or 79 with one or two conservative amino acid changes, and
F) a CDR3 amino acid sequence comprising SEQ ID NO:4, 8, 12, 16, 20, 24, 28, 32, 36, 40, 44, 48, 52, 56, 60, 64, 68, 72, 76, or 80; or SEQ ID NO:4, 8, 12, 16, 20, 24, 28, 32, 36, 40, 44, 48, 52, 56, 60, 64, 68, 72, 76, or 80 with one with one or two conservative amino acid changes.

4. The composition of claim 3, wherein said human CD6 binding molecule further comprises a linker which is attached to both said first SMVAD and said second SMVAD.

5. The composition of claim 3, wherein said one or more nucleic acid molecules comprise: i) a first nucleic acid sequence encoding said first SMVAD, and optionally further encoding a CH2 heavy chain constant region and/or a CH3 heavy chain constant region and ii) a second nucleic acid sequence encoding said second SMVAD, and optionally further encoding a CH2 heavy chain constant region and/or a CH3 heavy chain constant region.

6. The composition of claim 1, wherein said first SMVAD comprises the amino acid sequence shown in SEQ ID NO:1, 5, 9, 13, 17, 21, 25, 29, 33, 37, 41, 45, 49, 53, 57, 61, 65, 69, 73, or 77; or SEQ ID NO:1, 5, 9, 13, 17, 21, 25, 29, 33, 37, 41, 45, 49, 53, 57, 61, 65, 69, 73, or 77 with one, two, three, or four deletions and/or conservative amino acid changes.

7. The composition of claim 1, wherein said human CD6 binding molecule further comprises a CH2 heavy chain constant region and/or a CH3 heavy chain constant region.

8. The composition of claim 1, wherein said CH2 and/or CH3 heavy chain constant regions are camelid, humanized, or human.

9. The composition of claim 1, wherein said human CD6 binding molecule is conjugated to a cytotoxic agent, and optionally wherein said cytotoxic agent comprises Monomethyl auristatin (MMAE).

10. The composition of claim 1, further comprising a physiologically tolerable buffer.

11. The composition of claim 1, wherein said composition comprises said one or more nucleic acid molecules, and optionally the composition further comprises an expression vector, and wherein said one or more nucleic acid sequences are present in said expression vector.

12. The composition of claim 1, wherein said composition comprises said human CD6 binding molecule.

13. The composition of claim 1, wherein:
said CDR1 amino acid sequence comprises SEQ ID NO:2, 6, 10, 14, 18, 22, 26, 30, 34, 38, 42, 46, 50, 54, 58, 62, 66, 70, 74, or 78;
said CDR2 amino acid sequence comprises SEQ ID NO:3, 7, 11, 15, 19, 23, 27, 31, 35, 39, 43, 47, 51, 55, 59, 63, 67, 71, 75, or 79, and
said CDR3 amino acid sequence comprises SEQ ID NO:4, 8, 12, 16, 20, 24, 28, 32, 36, 40, 44, 48, 52, 56, 60, 64, 68, 72, 76, or 80.

14. A method of treating or preventing a T-cell related disease or condition comprising:
treating a subject with a composition comprising a human Cluster of Differentiation 6 (CD6) binding molecule, or an expression vector comprising said one or more nucleic acid molecules encoding said CD6 binding molecule, as recited in any of claims 1-13, and
wherein said subject has, or is suspected to develop, a T-cell related disease or condition.

15. The method of claim 14, wherein said T-cell related disease comprises cancer, and optionally wherein said cancer comprises T-cell lymphoma.

16. The method of claim 14, wherein said T-cell related disease comprises acute respiratory distress syndrome (ARDS), cytokine-release syndrome in a Covid-19 subject, or acute graft vs host disease (aCGDH).

17. The method of claim 14, wherein said T-cell related disease comprises lupus nephritis, uncontrolled asthma, psoriasis, or multiple schlerosis.

18. The method of claim 14, wherein said first SMVAD further comprises four Framework regions, wherein said four Framework regions are camelid, humanized, or human Framework regions.

19. The method of claim 14, wherein said human CD6 binding molecule further comprises a second SMVAD that comprises:
D) a CDR1 amino acid sequence comprising SEQ ID NO:2, 6, 10, 14, 18, 22, 26, 30, 34, 38, 42, 46, 50, 54, 58, 62, 66, 70, 74, or 78; or SEQ ID NO:2, 6, 10, 14, 18, 22, 26, 30, 34, 38, 42, 46, 50, 54, 58, 62, 66, 70, 74, or 78; with one with one or two conservative amino acid changes,
E) a CDR2 amino acid sequence comprising SEQ ID NO:3, 7, 11, 15, 19, 23, 27, 31, 35, 39, 43, 47, 51, 55, 59, 63, 67, 71, 75, or 79; or SEQ ID NO:3, 7, 11, 15, 19, 23, 27, 31, 35, 39, 43, 47, 51, 55, 59, 63, 67, 71, 75, or 79 with one or two conservative amino acid changes, and
F) a CDR3 amino acid sequence comprising SEQ ID NO:4, 8, 12, 16, 20, 24, 28, 32, 36, 40, 44, 48, 52, 56, 60, 64, 68, 72, 76, or 80; or SEQ ID NO:4, 8, 12, 16, 20, 24, 28, 32, 36, 40, 44, 48, 52, 56, 60, 64, 68, 72, 76, or 80 with one with one or two conservative amino acid changes.

20. The method of claim 19, wherein said human CD6 binding molecule further comprises a linker which is attached to both said first SMVAD and said second SMVAD.

21. The method of claim 19, wherein said one or more nucleic acid molecules comprise: i) a first nucleic acid sequence encoding said first SMVAD, and optionally further encoding a CH2 heavy chain constant region and/or a CH3 heavy chain constant region and ii) a second nucleic acid sequence encoding said second SMVAD, and optionally further encoding a CH2 heavy chain constant region and/or a CH3 heavy chain constant region.

22. The method of claim 14, wherein said first SMVAD comprises the amino acid sequence shown in SEQ ID NO:1, or SEQ ID NO:1 with one, two, three, or four deletions and/or conservative amino acid changes.

23. The method of claim 14, wherein said human CD6 binding molecule further comprises a CH2 heavy chain constant region and/or a CH3 heavy chain constant region.

24. The method of claim 14, wherein said CH2 and/or CH3 heavy chain constant regions are camelid, humanized, or human.

25. The method of claim 14, wherein said human CD6 binding molecule comprises at least an antigen binding portion of Clone 2G1 CD6 nanobody.

26. The method of claim 14, wherein said composition further comprises a physiologically tolerable buffer.

27. The method of claim 14, wherein said composition comprises said expression vector, and wherein said one or more nucleic acid sequences are present in said expression vector.

28. The method of claim 14, wherein said composition comprises said human CD6 binding molecule.

29. The method of claim 14, wherein:
said CDR1 amino acid sequence comprises SEQ ID NO:2, 6, 10, 14, 18, 22, 26, 30, 34, 38, 42, 46, 50, 54, 58, 62, 66, 70, 74, or 78;
said CDR2 amino acid sequence comprises SEQ ID NO:3, 7, 11, 15, 19, 23, 27, 31, 35, 39, 43, 47, 51, 55, 59, 63, 67, 71, 75, or 79, and
said CDR3 amino acid sequence comprises SEQ ID NO:4, 8, 12, 16, 20, 24, 28, 32, 36, 40, 44, 48, 52, 56, 60, 64, 68, 72, 76, or 80.

30. A method of detecting human Cluster of Differentiation 6 (CD6) in a sample comprising:
a) contacting a sample with the human CD6 binding molecule of any of claims 1-13, wherein said sample is suspected of containing human CD6, and wherein said human CD6 binding molecule forms a complex with said human CD6 if present in said sample; and
b) detecting the presence or absence of said complex in said sample.

31. The method of claim 30, wherein said sample is from a subject that has, or is suspected to develop, a T-cell related disease or condition.

32. The method of claim 30, wherein said human CD6 binding molecule comprises a detectable label.

33. The method of claim 30, further comprising contacting said sample with a conjugate molecule capable of binding to said human CD6 binding molecule, wherein said conjugate molecule comprises a detectable label.
